(19) United States
(12) Patent Application Publication
(10) Pub. No.: US 2024/0157118 A1
(43) Pub. Date: May 16, 2024

(54) BLOOD PUMP WITH THREE DIMENSIONAL ACTIVE ELECTROMAGNETIC SUSPENSION

(71) Applicant: Cardiobionic Pty Ltd, Caringbah, NSW (AU)

(72) Inventors: Peter Joseph Ayre, Frenchs Forest, NSW (AU); Reginald Brian Haddrell, Cronulla, NSW (AU); Ross James Dean, Cronulla, NSW (AU)

(73) Assignee: Cardiobionic Pty Ltd, Caringbah, NSW (AU)

(21) Appl. No.: 18/552,501

(22) PCT Filed: Mar. 25, 2022
(86) PCT No.: PCT/AU2022/050273

§ 371 Date: Sep. 26, 2023

(30) Foreign Application Priority Data
Mar. 26, 2021 (AU) 2021900895
Jul. 7, 2021 (AU) 2021902073
Dec. 13, 2021 (AU) 2021904037

(51) Int. Cl. A61M 60/822; A61M 60/178; A61M 60/221; A61M 60/422; A61M 60/538; A61M 60/806; A61M 60/816

(52) U.S. Cl. CPC A61M 60/822; A61M 60/178; A61M 60/221; A61M 60/422; A61M 60/538; A61M 60/806; A61M 60/816; A61M 2205/0272; A61M 2205/3317; A61M 2205/3365

(57) ABSTRACT

The invention relates to a rotary blood pump including a housing having an internal chamber, a blood inlet port and a blood outlet port, a rotor including a plurality of blades and being adapted to rotate within the chamber. The pump includes a bearing system for controlling the position of the rotor wherein the bearing system includes one or more permanent magnets embedded in the rotor and one or more electromagnetic field inducing means embedded in the housing. The magnets embedded in the rotor are influenced by the electromagnetic field inducing means embedded in the housing for controlling the position of the rotor relative to the internal chamber of the housing and for driving rotation of the impeller within the chamber of the housing.

### TECHNICAL FIELD

The present invention relates to a rotary blood pump. In particular, the invention relates to a rotary blood pump including a rotor or impeller located within a pumping chamber and a drive system for driving rotation of the impeller within the chamber and thereby circulating blood within the circulatory system. The invention is adapted for use in heart assist devices and systems commonly referred to as ventricular assist devices including associated sensors, controls systems and power supplies.

### BACKGROUND

Various rotary blood pumps have been developed to assist or to supplement the function of the human heart in circulating blood within the circulatory system. Rotary blood pumps are commonly found in ventricular assist devices (VADs). These devices are typically used as an interim measure with heart transplant being the ultimate long-term solution. However, these devices are increasingly being considered as a long-term therapy meaning the patient would live with the device for the rest of their life.

A rotary blood pump has an impeller disposed within a pumping chamber of a pump housing. Blood is delivered via an axial inlet of the housing and is pumped by the impeller to a radial outlet. The impeller is rotatably driven within the pumping chamber by a drive system, such as one or more stationary drive magnets in the impeller that are rotatably driven by an electromagnet in the housing.

An important consideration with continuous flow rotary pumps is the method used to suspend the rotor or impeller. Some existing blood pumps use solid bearings whereas others employ either magnetic levitation (“maglev”) or hydrodynamic suspension. Many such existing blood pumps contain only one moving part, namely the rotor or impeller.

Rotation of the rotor or impeller is via a brushless motor arrangement. A brushless motor uses a direct current (DC) electric power supply and an electronic closed loop controller to switch DC currents to motor windings in a stator that produces magnetic fields which effectively rotate in space. Permanent magnets mounted in the rotor are influenced by the rotating magnetic fields which drives the rotation of the rotor. The controller adjusts the phase and amplitude of the DC current pulses to control the speed and torque of the motor.

A need exists for a rotary blood pump that can exhibit effective control over the position and movement of the rotor or impeller. For example by controlling movement of the rotor or impeller relative to the chamber containing the rotor or impeller and preferably also by controlling tolerances therebetween. A need also exists for rotary blood pumps that can ameliorate blood damage such as haemolysis due to hydrodynamic shearing and associated shear stress forces experienced by blood cells. A need also exists for rotary blood pumps that can ameliorate blood stasis causing blood clots (thrombosis).

The discussion of the background to the invention included herein including reference to documents, acts, materials, devices, articles and the like is included to explain the context of the present invention. This is not to be taken as an admission or a suggestion that any of the material referred to was published, known or part of the common general knowledge in Australia or in any other country as at the priority date of any of the claims.

### SUMMARY OF INVENTION

In one aspect, the present invention provides a blood pump including:
a housing having an internal chamber, a blood inlet port and a blood outlet port;
a rotor including a plurality of blades and being adapted to rotate within the chamber wherein blood received into the chamber via the inlet port is directed by the blades of the rotor out of the chamber via the outlet port and
a bearing system for controlling the position of the rotor relative to the internal chamber of the housing, the bearing system including:
   one or more permanent magnets embedded in the rotor;
   one or more electromagnetic field inducing means embedded in the housing;
wherein the permanent magnets embedded in the rotor are influenced by the electromagnetic field inducing means embedded in the housing for controlling the position of the rotor relative to the internal chamber of the housing and for driving rotation of the impeller within the chamber of the housing.

Embodiments of the invention are advantageous in that the electromagnetic field inducing means, preferably coils, are operable as commutating coils for driving rotation of the impeller and for electromagnetically positioning, such as by levitating the impeller, in three dimensions (i.e. in three axes X, Y, Z) such as by controlling the current provided to each of the coils.

In embodiments, the pump is configured so that the electromagnetic field inducing means embedded in the housing are each selectively provided with an electrical current to selectively generate one or more magnetic fields for controlling the relative position of the rotor within the chamber in three-dimensions.

Embodiments of the invention are advantageous in that the electromagnetic field inducing means are operable for controlling the position of the rotor within the housing (i.e. for levitating and/or for orientation of the rotor and clearance around the rotor) and for driving rotation of the rotor (i.e. commutation). Increasing the functionality of the electromagnetic field inducing means avoids the requirement for separate components to perform the levitation and commutation functions enables a smaller less complicated pump.

In embodiments, the permanent magnets and the electromagnetic field inducing means control the position of the rotor within the internal chamber of the housing in three dimensions.

Embodiments of the invention are advantageous in that the electromagnetic field inducing means are operable for controlling the position of the rotor within the housing in three dimensions and for driving rotation of the rotor (i.e. commutation) thereby enabling a smaller less complicated pump.

In embodiments, the permanent magnets and the electromagnetic field inducing means control the position of the rotor within the internal chamber of the housing in an axial direction and in any direction in a plane normal to the axial direction.

In embodiments, the permanent magnets and the electromagnetic field inducing means are disposed radially about the rotor.

In embodiments, the permanent magnets are axially spaced apart from the electromagnetic field inducing means.

In embodiments, the permanent magnets and the electromagnetic field inducing means are oriented at an angle between the axis of rotation of the rotor and the normal to the axis of rotation of the rotor.

Preferably, the permanent magnets and the electromagnetic field inducing means are oriented at an incline or at a decline to a horizontal plane that is perpendicular to a central longitudinal axis of the chamber.

Preferably, the permanent magnets and the electromagnetic field inducing means are oriented at an incline or at a decline angle of between about 5 degrees to 30 degrees or between about 10 to 20 degrees or about 15 degrees.

Preferably, the electromagnetic field inducing means includes one or more wire coils producing magnetic fields.

Preferably, the electromagnetic field inducing means includes wire coils disposed radially about the central longitudinal axis of the housing at equal angularly spaced apart intervals.

Preferably, six of the wire coils are disposed radially about the rotor.

In embodiments, the electromagnetic field inducing means includes upper electromagnetic field inducing means and lower electromagnetic field inducing means spaced apart in the direction of the central longitudinal axis of the housing.

Preferably, the upper electromagnetic field inducing means and the lower electromagnetic field inducing means are respectively oriented at an incline and at a decline by equivalent angles from a horizontal plane of the impeller and are thereby oriented symmetrically about the horizontal plane.

In embodiments, the upper electromagnetic field inducing means are embedded in a downwardly sloping portion of an upper wall of the chamber and the lower electromagnetic field inducing means are embedded in an upwardly sloping portion of a lower wall of the chamber.

Preferably, an electric current passed through each one of the wire coils is controlled independently to control the resulting magnetic fields produced thereby, wherein the interaction of the magnetic fields of the permanent magnets and of the wire coils induces forces acting between the impeller and the housing.

In embodiments, a resultant force between the impeller and the housing controls the relative position of the impeller within the chamber of the housing.

In embodiments, a resultant force between the impeller and the housing drives rotation of the impeller within the chamber of the housing.

In embodiments, the blood pump includes a rotor position detection system for detecting the position of the rotor relative to the internal chamber of the housing,

In embodiments, the position detection system includes:
one or more magnetic field sensors embedded in the housing;
wherein the magnetic field sensors are influenced by the permanent magnets embedded in the rotor for determining the position of the rotor relative to the internal chamber of the housing.

Preferably, the magnetic field sensors are disposed radially about the rotor.

Preferably, the magnetic field sensors are disposed at angularly spaced apart intervals.

Preferably, six of the magnetic field sensors are disposed radially about the rotor at equal angularly spaced apart intervals.

In embodiments, the position detection system includes the electromagnetic field inducing means being operable for detecting the position of the impeller within the chamber of the housing.

Preferably, the electromagnetic field inducing means are operable for detecting Back emf and thereby determining any imbalance of the impeller within the chamber of the housing.

Preferably, the rotor position detection system detects the relative position of the rotor within the internal chamber of the housing in three dimensions including the axial direction and any direction in a plane normal to the axial direction.

In embodiments, a stator is embedded in the housing comprised of the one or more electromagnetic field inducing means embedded in the housing, wherein the permanent magnets embedded in the rotor are influenced by the magnetic field generated by the one or more electromagnetic field inducing means embedded in the housing to thereby drive the rotation of the rotor within the chamber.

In embodiments, three of the upper electromagnetic field inducing means are spaced apart at equal angularly spaced apart intervals of preferably 120 degrees and three of the lower electromagnetic field inducing means are spaced apart at equal angularly spaced apart intervals of preferably 120 degrees.

In embodiments, the upper electromagnetic field inducing means are positioned at angularly offset positions relative to the lower electromagnetic field inducing means, wherein the offset between the upper electromagnetic field inducing means and the lower electromagnetic field inducing means is preferably 60 degrees.

In embodiments, each one of the blades of the rotor includes at least one and preferably two of the embedded permanent magnets.

In embodiments, the embedded permanent magnets are oriented at an incline or at a decline to a horizontal plane that is perpendicular to a central longitudinal axis of the impeller, wherein the permanent magnets are oriented at an incline or a decline angle of between about 20 to 50 degrees or between about 30 to 40 degrees or about 36 degrees to the horizontal plane, preferably parallel to the incline of the impeller

Preferably, the one or more electromagnetic field inducing means are embedded in the housing in an orientation that is complementary to the orientation of the permanent magnets embedded in the rotor blades.

In embodiments, a controller is operable to control the speed of the pump to thereby control the output of the pump.

In embodiments, the controller is operable to control the electromagnetic fields induced by the one or more electromagnetic field inducing means embedded in the housing for controlling the position of the rotor relative to the internal chamber of the housing.

Preferably, the controller is configured to selectively provide electrical current to the electromagnetic field inducing means embedded in the housing to selectively generate one or more magnetic fields for controlling the relative position of the rotor within the chamber in three-dimensions

In embodiments, the controller receives a signal from the one or more magnetic field sensors or the electromagnetic field inducing means embedded in the housing and determines the position of the rotor relative to the internal chamber of the housing.

Preferably, the controller receives a signal from the magnetic field inducing means embedded in the housing for determining the position of the rotor relative to the internal chamber of the housing.

In another aspect, the invention includes a method for controlling a position of a rotor relative to an internal chamber of a housing of a blood pump, the method including:
receiving in a controller signals from sensors indicative of the relative position of an impeller within a blood pump housing;
processing the signals in the controller to determine the relative position of the impeller within the blood pump housing; and
providing an electrical current to one or more electromagnetic field inducing means embedded in the housing to thereby generate one or more magnetic fields, wherein the one or more magnetic fields influence permanent magnets embedded in the rotor for controlling the position of the rotor relative to the internal chamber of the housing.

In embodiments, the method includes selectively providing an electrical current to the one or more electromagnetic field inducing means embedded in the housing to selectively generate one or more magnetic fields for controlling the relative position of the rotor within the internal chamber of the housing in three dimensions. Preferably, the three dimensions include a direction of the axis of rotation of the rotor and any direction is in a plane normal to the axis of rotation of the rotor.

Preferably, the method includes receiving signals from a plurality of magnetic field sensors embedded in the housing that are induced by the permanent magnets embedded in the rotor and determining from the signals the position of the rotor relative to the internal chamber of the housing.

These and other aspects and embodiments of the invention will become apparent from the foregoing summary of the drawings and the detailed description.

### BRIEF DESCRIPTION OF DRAWINGS

The present invention will now be described in more detail with reference to embodiments of the invention illustrated in the figures, wherein:

FIG. 1a illustrates a schematic representation of a side view of a blood pump according to an embodiment of the invention including a housing containing a rotor;

FIG. 1b illustrates schematically a top view of the blood pump of FIG. 1 and the relative position of electromagnetic field inducing means embedded in a housing of the blood pump above the rotor;

FIG. 1c illustrates schematically a bottom view of the blood pump of FIG. 1 and the relative position of electromagnetic field inducing means embedded in the housing of the blood pump below the rotor;

FIG. 1d illustrates schematically a top view of the rotor of the blood pump of FIG. 1 illustrating the relative polarity of permanent magnets within blades of the rotor;

FIGS. 2a and 2b illustrate schematically controllers for controlling blood pumps according to embodiments of the invention;

FIG. 3 illustrates a method for controlling a position of a rotor or impeller relative to an internal chamber of a housing of a blood pump in accordance with an embodiment of the invention;

FIG. 4 illustrates a frontal cross section view of a blood pump according to an embodiment of the invention;

FIG. 5 illustrates a perspective view of the blood pump of FIG. 4;

FIG. 6 illustrates a top view of the blood pump of FIG. 4;

FIG. 7 illustrates a perspective view of an impeller of the blood pump of FIG. 4;

FIG. 8 illustrates a perspective view of the impeller of the blood pump of FIG. 4 viewed from a different aspect to the view of FIG. 7;

FIG. 9 illustrates a perspective view of permanent magnets that are within the blades of the impeller of the blood pump of FIG. 4;

FIG. 10 illustrates a side view of a frontal cross section of the impeller of the blood pump of FIG. 4; and

FIG. 11 illustrates a top view of the impeller of the blood pump of FIG. 4.

These and other aspects and embodiments of the invention will become apparent from the foregoing summary of the drawings and the detailed description.

### DETAILED DESCRIPTION

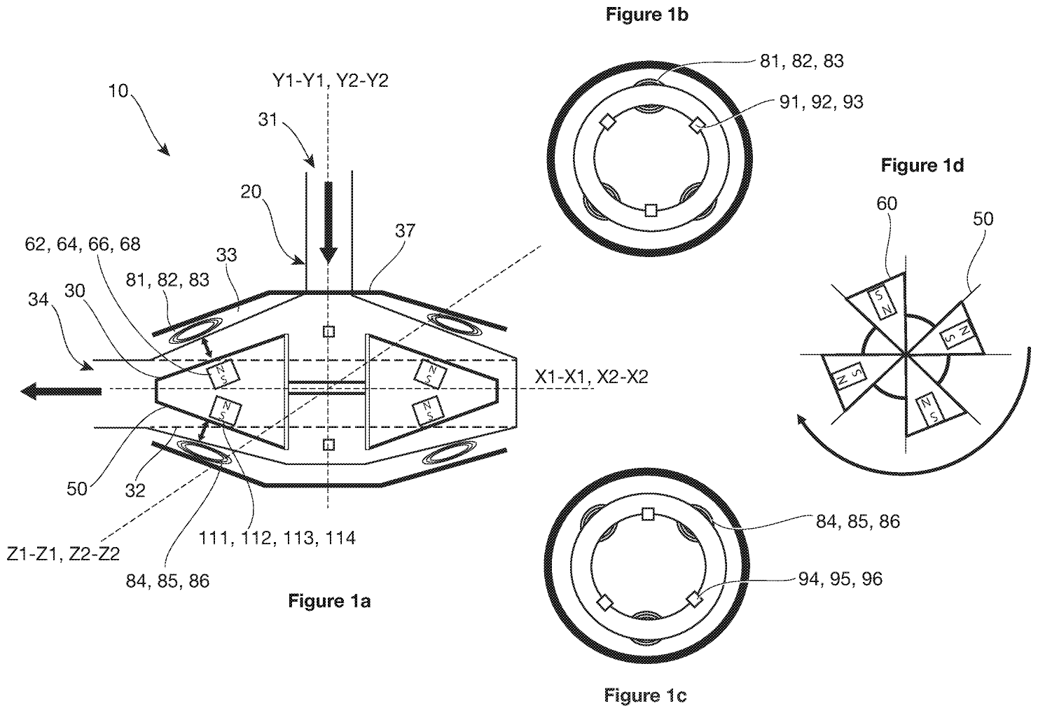

Referring to the embodiment of FIG. 1a, the invention relates to a blood pump 10 adapted for providing mechanical circulatory support for use in the management of advanced heart failure. In embodiments, the blood pump 10 is a rotary blood pump including a housing 20 with an internal chamber 30, a blood inlet port 31 and a blood outlet port 34. Located within the internal chamber is a rotor 50 including a number of blades 60. The rotor 50 is adapted to rotate within the chamber 30 and blood received into the chamber 30 via the inlet port 31 is directed by the blades 60 of the rotating rotor 50 out of the chamber 30 via the outlet port 34.

The pump 10 includes a bearing system for controlling the position of the rotor 50 relative to the internal chamber 30 of the housing 20. The bearing system includes one or more permanent magnets 62, 64, 66, 68, 111, 112, 113, 114 embedded in the rotor 50 as well as one or more electromagnetic field inducing means 81, 82, 83, 84, 85, 86 embedded in the housing 20. The permanent magnets 62, 64, 66, 68, 111, 112, 113, 114 embedded in the rotor 50 are influenced by the electromagnetic field inducing means 81, 82, 83, 84, 85, 86 embedded in the housing 20 for driving rotation of the rotor 50 and for controlling the position of the rotor 50 relative to the internal chamber 30 of the housing 20.

Referring to FIG. 1a, the pump 10 also includes a rotary drive system that is adapted to drive the rotation of the impeller 51 about a central axis Y2-Y2 thereof. Rotation of the impeller 51 within the chamber 30 results in blood received into the chamber 30 via the inlet port 31 being directed by the blades 60 of the impeller 51 out of the chamber 30 via the outlet port 34.

The electromagnetic field inducing means 81, 82, 83, 84, 85, 86 embedded in the housing 20 are each comprised of a wire coil 81a, 82a, 83a, 84a, 85a, 86a that is configured to generate a magnetic field when an electric current is passed therethrough. The permanent magnets 62, 64, 66, 68, 111, 112, 113, 114 embedded in the rotor 50 are influenced by the magnetic fields generated by the wire coils 81a, 82a, 83a, 84a, 85a, 86a to thereby drive the rotation of the impeller 51 within the chamber 30. As described in further detail below, the pump 10 is configured so that the electromagnetic field inducing means 81, 82, 83, 84, 85, 86 embedded in the housing 20 are each selectively provided with an electrical current to selectively generate one or more magnetic fields for controlling the relative position of the rotor 50 within the internal chamber 30 in three-dimensional space.

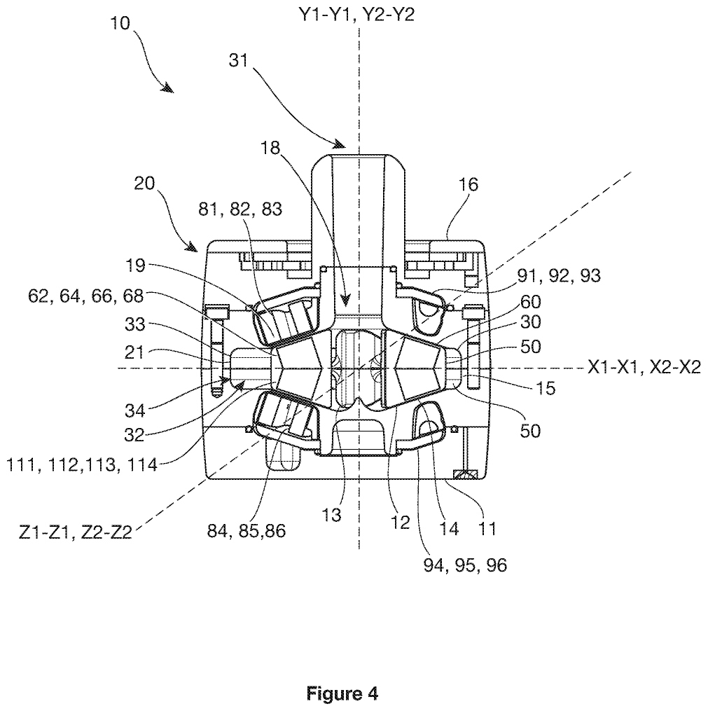

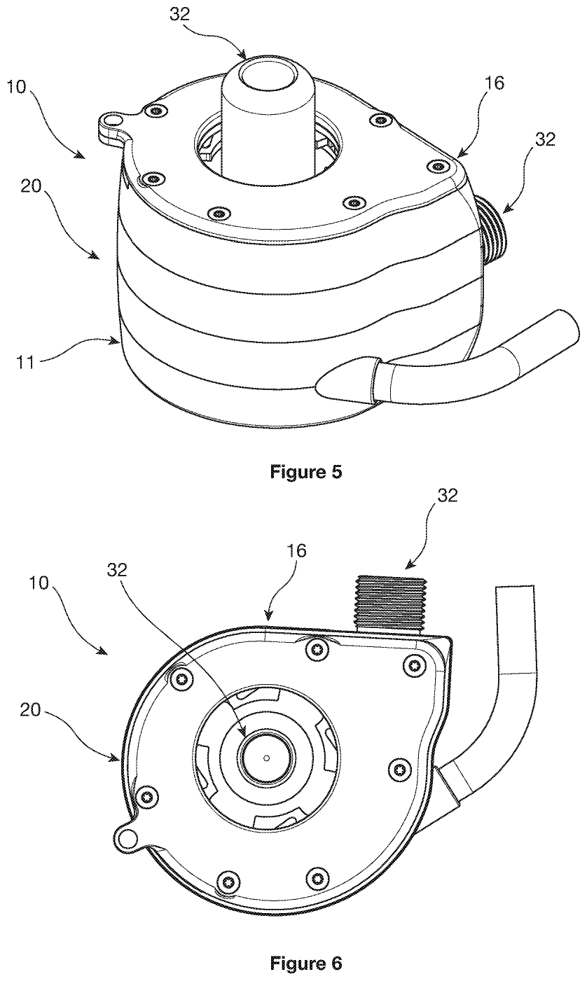

Referring to the embodiment of FIGS. 4 to 6 the inlet 31 is tubular in shape and is moulded into the housing 20 and is in fluid communication with an internal volume 32 within the chamber 30. The inlet 31 is located at an upper region of the housing 20 and substantially axially with a central longitudinal axis of the housing 20. The inlet 31 is adapted for connection to an inlet tube (not shown) that is adapted to be connected to the left ventricle or at another location within the patient's circulatory system depending on the type of circulatory support required.

The outlet 34 is also tubular in shape and is moulded into the housing 20 and is in fluid communication with the internal volume 32 within the chamber 30. The outlet 34 is located at a lower region of the housing 20 and substantially perpendicularly and tangentially relative to the chamber 30 and the longitudinal axis thereof. The outlet 34 is adapted for connection to an outlet tube (not shown) that is adapted to be connected to the aorta or at another location within the patient's circulatory system depending on the type of circulatory support required.

Referring to FIGS. 4 to 6, the housing 20 is formed out of a bottom moulded section 11 and a top moulded section 16 that are brought together to define the chamber 30. The bottom moulded section 11 defines a lower wall 12 of the chamber 30. The lower wall 12 is comprised of a central wall portion 13, an upwardly sloping intermediate wall portion 14 and an upstanding peripheral wall portion 15. The top moulded section 16 defines a top wall 17 that opposes the lower wall 12. The top wall 17 has a central opening 18 and an intermediate downwardly sloping wall 19 located radially outwards from the central opening 17 and a downwardly depending peripheral wall portion 21. The central opening 17 is in fluid communication with the inlet 31 for blood to enter the chamber 30. The upstanding peripheral wall portion 15 of the bottom moulded section 11 includes an outlet opening 22 in fluid communication with the blood outlet port 34 for blood to exit the chamber 30. The upstanding peripheral wall portion 15 and the downwardly depending peripheral wall portion 21 together define an annular side wall 33 extending between the bottom wall 12 and the top wall 17. The resulting chamber 30 is substantially symmetrical about a horizontal plane, that is a plane that is perpendicular to a central longitudinal axis Y1-Y1 of the chamber 30.

The rotor 50 is in the form of an impeller 51 and is rotatably supported within the chamber 30. The impeller 51 is configured for pumping blood received in the inlet 32 to expel the blood out of the outlet 34. The impeller 51 is rotatably driven by a drive system. As described herein, the drive system is configured to rotate the impeller 51 about the central longitudinal axis Y1-Y1 of the chamber 30 such that the impeller 51 pumps blood from the inlet 32 to the outlet 34. The impeller 51 is rotatably supported within the chamber 30 by a bearing system. As described herein, the bearing system assists in positioning the impeller 51 within the chamber 30 such that the impeller 51 rotates about the central longitudinal axis Y1-Y1 without touching the walls of the chamber, namely the bottom wall 12 or the upper wall 17 of the chamber 30, or for that matter the peripheral side walls 15, 21 of the chamber 30.

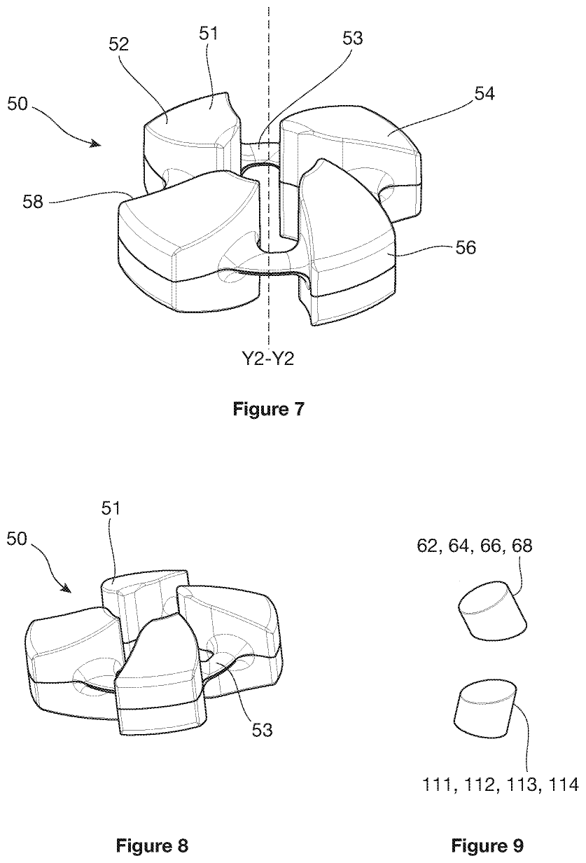

Referring to FIG. 7, the impeller 51 has a set of blades 52, 54, 56, 58 that are interconnected by annular connection members 53 extending between and connected to adjacent pairs of the blades 52, 54, 56, 58. The blades 52, 54, 56, 58 of the rotating impeller 51 are adapted to accelerate blood that is received axially via the inlet 32 into the chamber 30 in a radially outwards direction towards the side wall 33. The side wall 33 may be shaped to form a volute chamber that directs the accelerated blood out of the chamber 30 via the outlet 34.

With reference to the embodiments of FIGS. 1a and 4 the features and operation of the impeller drive and bearing system will now be described. A small protuberance 39 is located within the chamber 30 upstanding from the central wall portion 13 of the bottom wall 12 in alignment with the longitudinal axis Y1-Y1. The impeller 51 includes a central opening 59 that is aligned coaxially with the longitudinal axis Y1-Y1 of the chamber 51.

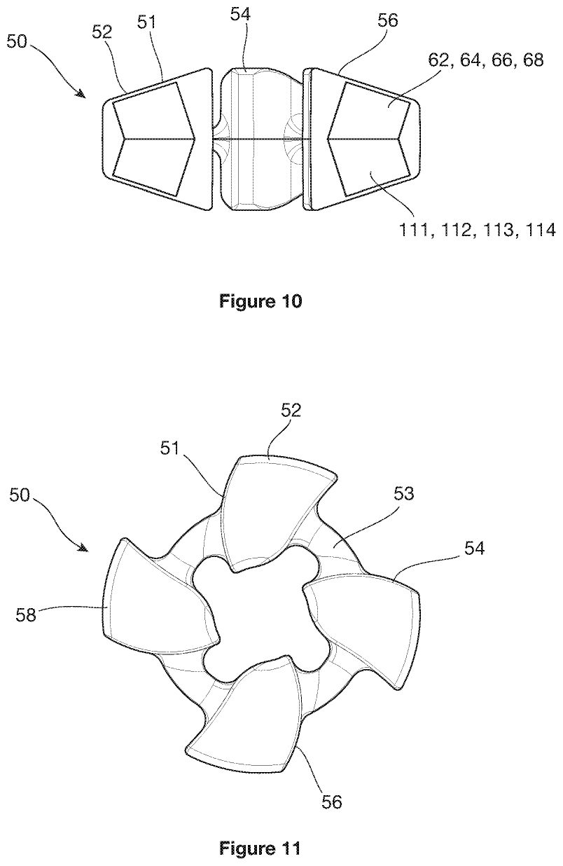

Referring to FIGS. 1a, 4, 9 and 10, the blades 52, 54, 56, 58 of the impeller 51 include a set of upper permanent magnets 62, 64, 66, 68 and a set of lower permanent magnets 111, 112, 113, 114 located radially about the impeller 51.

The upper permanent magnets 62, 64, 66, 68 are located immediately adjacent to an upwardly facing edge or surface of each blade 52, 54, 56, 58. The upper permanent magnets 62, 64, 66, 68 are oriented at an incline to a horizontal plane through the impeller 51, wherein the horizontal plane is aligned with an axis X2-X2 that is perpendicular to the central axis Y2-Y2 of the impeller 51. In embodiments, the permanent magnets 62, 64, 66, 68 are oriented at an incline to the horizontal plane of the impeller 51 aligned with the axis X2-X2 at an angle of between about 20 to 50 degrees or between about 30 to 40 degrees or about 36 degrees.

The lower permanent magnets 111, 112, 113, 114 are located immediately adjacent to a downwardly facing edge or surface of each blade 52, 54, 56, 58. The lower permanent magnets 111, 112, 113, 114 are oriented at a decline to the horizontal plane through the impeller 51, namely the plane aligned with the axis X2-X2. In embodiments, the lower permanent magnets 111, 112, 113, 114 are oriented at a decline to the horizontal plane of the impeller 51 aligned with the axis X2-X2 of between about 20 to 50 degrees or between about 30 to 40 degrees or about 36 degrees.

The upper permanent magnets 62, 64, 66, 68 and the lower permanent magnets 111, 112, 113, 114 are respectively oriented at an incline and at a decline to the horizontal plane of the impeller 51 aligned with the horizontal axis X2-X2 through the impeller 51 by equivalent angles and are thereby oriented symmetrically about the horizontal plane through the impeller 51.

Upper electromagnetic field inducing means 81, 82, 83 are embedded in the intermediate downwardly sloping portion 19 of the upper wall 17 of the chamber 30. Lower electromagnetic field inducing means 84, 85, 86 are embedded in the upwardly sloping intermediate wall portion 14 of the lower wall 12.

As mentioned above, the electromagnetic field inducing means 81, 82, 83, 84, 85, 86 are each comprised of a wire coil 81a, 82a, 83a, 84a, 85a, 86a that is configured to generate a magnetic field when an electric current is passed therethrough. The wire coils 81a, 82a, 83a, 84a, 85a, 86a are disposed radially about the chamber 30 within the housing 20.

The upper electromagnetic field inducing means 81, 82, 83 are spaced apart at equal angularly spaced apart intervals of preferably 120 degrees and the lower electromagnetic field inducing means 84, 85, 86 are spaced apart at equal angularly spaced apart intervals of preferably 120 degrees. The upper electromagnetic field inducing means 81, 82, 83 are positioned at angularly offset positions relative to the lower electromagnetic field inducing means 84, 85, 86. The offset between the upper electromagnetic field inducing means 81, 82, 83 and the lower electromagnetic field inducing means 84, 85, 86 is preferably 60 degrees.

In embodiments, the wire coils 81a, 82a, 83a, 84a, 85a, 86a are oriented at an incline to a horizontal plane of the chamber 30, namely a horizontal plane aligned with an axis X1-X1 that is perpendicular to the central longitudinal axis Y1-Y1 of the chamber 30. The upper wire coils 81a, 82a, 83a are oriented at an angle of incline to the horizontal plane of the chamber 30 of between about 20 to 50 degrees or between about 30 to 40 degrees or about 36 degrees. The lower wire coils 84a, 85a, 86a are oriented at an angle of decline to the horizontal plane of the chamber 30 of between about 20 to 50 degrees or between about 30 to 40 degrees or about 36 degrees.

The upper wire coils 81a, 82a, 83a and the lower wire coils 84a, 85a, 86a are respectively oriented at an incline and at a decline to the horizontal plane of the chamber 30 aligned with the axis of X1-X1 by equivalent angles and are thereby oriented symmetrically about the horizontal plane through the chamber 30.

As will be described in further detail below, the electric current passed through each one of the wire coils 81a, 82a, 83a, 84a, 85a, 86a is controlled independently to control the resulting magnetic field. As the impeller 51 rotates the upper permanent magnets 62, 64, 66, 68 and the lower permanent magnets 111, 112, 113, 114 pass through the magnetic fields associated with each one of the wire coils 81a, 82a, 83a, 84a, 85a, 86a. The interaction of the magnetic fields associated with the permanent magnets 62, 64, 66, 68, 111, 112, 113, 114 and the wire coils 81a, 82a, 83a, 84a, 85a, 86a induces forces acting between the impeller 51 and the housing 20. The resultant force between the impeller 51 and the housing 20 controls the relative position of the impeller 51 within the chamber 30 of the housing 20.

Because the permanent magnets 62, 64, 66, 68, 111, 112, 113, 114 and/or the wire coils 81a, 82a, 83a, 84a, 85a, 86a are oriented at inclined or declined angles to the horizontal planes of the chamber 30 and the impeller 51, and therefore also at inclined or declined angles to the central longitudinal axis Y1-Y1 of the chamber 30 and/or the axis of rotation Y2-Y2 of the impeller 51, the resultant force between the impeller 51 and the housing 20 includes a vector in the Y1-Y1 axis and a vector in any direction in the normal to the Y1-Y1 axis, namely in the X1-X1 axis and in a Z1-Z1 axis, as illustrated in FIGS. 1a and 4.

In the embodiments of FIGS. 1a and 4, the bearing systems are configured so that the resultant forces between the impeller 51 and the housing 20 control the location of the impeller 51 within the chamber 30 in three dimensions and/or axes, namely: 1) the central longitudinal axis Y1-Y1 of the chamber 30; 2) a first axis normal to the central longitudinal axis Y1-Y1 of the chamber 30 (which is also normal to the axis of rotation of the impeller 51), namely the X1-X1 axis; and 3) a second axis normal to central longitudinal axis Y1-Y1 of the chamber 30, namely the Z1-Z1 axis, that is perpendicular to both the Y1-Y1 and X1-X1 axes. The X1-X1 and Z1-Z1 axes lie in a plane, which is also the horizontal plane through the chamber 51 and represent movement in any direction in that plane. Thus, the position of the impeller 51 within the chamber 30 of the housing 20 can be controlled in three dimensions (i.e. X1-X1, Y1-Y1 and Z1-Z1).

In an embodiment, the orientation of the permanent magnets 62, 64, 66, 68, 111, 112, 113, 114 and/or the wire coils 81a, 82a, 83a, 84a, 85a, 86a at an angle, such as between about 20 to 50 degrees or between about 30 to 40 degrees or about 36 degrees, or alternatively between about 30 and 60 degree or about 45 degrees, to the central axis Y1-Y1 of the chamber 30 and/or the axis of rotation Y2-Y2 of the impeller 51 can also enable control of the relative position of the impeller 51 within the internal chamber 30 angularly to the central axis Y1-Y1 of the chamber 30 and/or the axis of rotation Y2-Y2 of the impeller 51. The angular position of the impeller 51 within the chamber 30 represents a tilt of the impeller 51 relative to the central axis Y1-Y1 of the chamber 30.

By controlling the location of the impeller 51 within the chamber 30 in three dimensions, clearance gaps or tolerances between the impeller 51 and the base 31 and side wall 33 of the chamber 30 can be controlled. Controlling movement of the impeller 51 relative to the chamber 30 and thereby controlling tolerances therebetween can ameliorate blood damage such as haemolysis due to hydrodynamic shearing and associated shear stress forces experienced by blood cells.

As illustrated in FIGS. 1a, 1b, 1c and 4, the upper electromagnetic field inducing means 81, 82, 83 are positioned radially about the central axis Y1-Y1 of the chamber 30 to form an upper yoke and the lower electromagnetic field inducing means 84, 85, 86 are positioned radially about the central axis Y1-Y1 of the chamber 30 to form a lower yoke. The upper and the lower yokes together support and maintain the orientation of the impeller 51 relative to the central axis Y1-Y1 of the chamber 30.

Referring to FIGS. 1a, 1b, 1c, 2a, 2b and 4, the blood pump 10 includes a rotor 50 or impeller 51 position detection system for detecting the position of the rotor or impeller 51 relative to the internal chamber 30 of the housing 20.

In embodiments, the position detection system includes one or more magnetic field sensors 91, 92, 93, 94, 95, 96 embedded in the housing 20. Preferably, as illustrated in FIGS. 1a and 1b, three of the magnetic field sensors 91, 92, 93 are located radially about the central longitudinal axis Y1-Y1 of the housing 20 preferably at 120 degree intervals and embedded in the upper intermediate inclined wall portion 19 of the housing 20 similarly to the electromagnetic field inducing coils 81a, 82a, 83a as described above. FIGS. 1a and 1b illustrates another three of the magnetic field sensors 94, 95, 96 are located radially about the central longitudinal axis Y1-Y1 of the housing 20 preferably at 120 degree intervals and embedded in the lower intermediate inclined wall portion 14 of the housing 20 similarly to the electromagnetic field inducing coils 84a, 85a, 86a.

In the embodiment illustrated in FIGS. 1a, 1b and 1c, the magnetic field sensors 91, 92, 93, 94, 95, 96 are located at the midpoints between adjacent pairs of the electromagnetic field inducing coils 81a , 82a, 83a, 84a, 85a, 86a. The magnetic field sensors 91, 92, 93, 94, 95, 96 are preferably electromagnetic coils.

Preferably the magnetic field sensors 91, 92, 93, 94, 95, 96 are influenced by the permanent magnets 62, 64, 66, 68 embedded in the blades 52, 54, 56, 58 of the impeller 51 for determining the position of the impeller 51 relative to the internal chamber 30 of the housing 20. In particular, the magnetic field sensors 91, 92, 93, 94, 95, 96 generate an electrical signal in response to the movement of the permanent magnets 62, 64, 66, 68 in the impeller 51 in the vicinity of the sensors 91, 92, 93, 94, 95, 96.

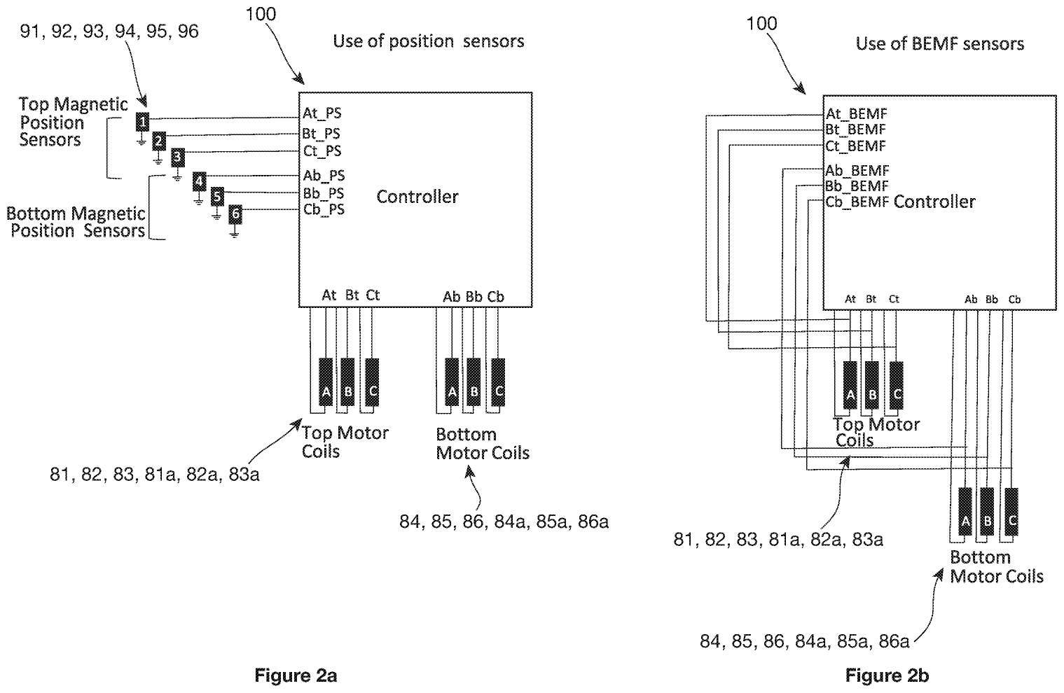

As illustrated in FIG. 2a, the signals generated by the sensors 91, 92, 93, 94, 95, 96 are received by a controller 100. The controller 100 includes a processor for processing the signals and for determining the relative position of the impeller 51 relative to the internal chamber 30 of the housing 20 in real time as the impeller 51 rotates. The controller 100 is operable to determine the relative position of the impeller 51 within the internal chamber 30 of the housing 20 in any one or more of the axial direction (i.e. the Y-Y axis) or in any direction normal to the axial direction (i.e. in the X-X axis and Z-Z axis).

In an embodiment, the signals generated by the sensors 91, 92, 93, 94, 95, 96 and received by the controller 100 can be processed to determine the angular position of the impeller 51 relative to the central axis Y1-Y1 of the chamber 30. Thus, the tilt of the impeller 51 relative to the central axis Y1-Y1 of the chamber 30 can be determined in real time.

The controller 100 is operable to determine the impeller position information by calculating an error between the top sensors 91, 92, 93 and the bottom sensors 94, 95, 96 indicating an imbalance in the impeller position relative to the central longitudinal axis Y1-Y1 of the chamber 30. The impeller position information is fed into an algorithm to adjust the impeller position in real time by modulating the power provided to the electromagnetic field inducing coils 81a, 82a, 83a, 84a, 85a, 86a.

Referring to FIG. 2a, there is illustrated a schematic representation of an embodiment in which the controller 100 and the pump 10 including the three upper impeller position sensors 91, 92, 93 (identified as Phase A top, B top & C top) and three lower impeller position sensors 94, 95, 96 (identified as Phase A bottom, B bottom & C bottom). The position of the impeller 51 in three dimensions X, Y, Z is measured using the six impeller position sensors 91, 92, 93, 94, 95, 96 comprised of the three upper sensors 91, 92, 93 and three lower sensors 94, 95, 96. The chamber 30 of the pump 10 is symmetrical and therefore error between the top sensors 91, 92, 93 and bottom sensors 94, 95, 96 yields an imbalance in the position of the impeller 51. The error information is fed into an algorithm to adjust the position of the impeller 51 in real-time by varying the current individually in each of the electromagnetic field inducing motor coils 81a, 82a, 83a, 84a, 85a, 86a (i.e. top and bottom motor coil phases A, B, C). The magnetic sensors can be separate to the coils or the undriven motor coil can be also used as a sensor.

Accordingly, embodiments of the invention are advantageous in that the electromagnetic field inducing coils 81a, 82a, 83a, 84a, 85a, 86a are operable as commutating coils for driving rotation of the impeller 51 and for magnetically levitating the impeller 51 in three axes X, Y, Z by controlling the current provided to each of the coils 81a, 82a, 83a, 84a, 85a, 86a.

From this sensor and similarly other sensing during rotation the location or position of the impeller 51 can be calculated. From this information the impeller position is known at any time. Impeller position correction and centring is done by controlling the magnetic field in the motor drive coils or current such that if for example one of the blades of the impeller needs lifting then the power provided to one of the upper coils can be increased.

In embodiments, the controller 100 is operable to implement control of the position of the impeller 51 within the chamber 30 according to the following algorithm:

Impeller offset X=(Xa*Sensor A error+Xb*Sensor B error+Xc*Sensor C error)

Impeller offset Y=(Ya*Sensor A error+Yb*Sensor B error+Yc*Sensor C error)

Impeller offset Z=(Za*Sensor A error+Zb*Sensor B error+Zc*Sensor C error),

Where Impeller offset (X, Y, Z) are the offset errors of the impeller from the central point (0,0,0), where the impeller centre is at (0,0,0) when perfectly balanced and the following are the scaling constants:

$$Uxyz = [ \begin{matrix}Xa , Xb , Xc \\ Ya , Yb , Yc \\ Za , Zb , Zc\end{matrix} ]$$

The Coil currents offset between the top and bottom coil accordingly are:

Phase A Current offset=A*Impeller offset X+B*Impeller offset Y+C*Impeller offset Z

Phase B Current offset=D*Impeller offset X+E*Impeller offset Y+F*Impeller offset Z

Phase C Current offset=G*Impeller offset X+H*Impeller offset Y+!Impeller offset Z

$$Current scale matrix = [ \begin{matrix}A , B , C \\ D , E , F \\ G , H , H\end{matrix} ]$$

The Current for each coil are as follows:

Phase A Top coil current=Phase A current+Phase A Current offset

Phase B Top coil current=Phase B current+Phase B Current offset

Phase C Top coil current=Phase C current+Phase C Current offset

Phase A Bottom coil current=Phase A current−Phase A Current offset

Phase B Bottom coil current=Phase B current−Phase B Current offset

Phase C Bottom coil current=Phase C current−Phase C Current offset

All of the above assume a constant motor voltage and as an example, the current is set by pulse width modulation. Both constant matrices may be pre-set or trained by an operator and updated through learning algorithms such as Neuro-fuzzy logic.

In embodiments, both constant matrices detailed above include values that are predetermined or that are dynamically determined or a combination of both, namely predetermined and then dynamically revised. The predetermined values are preferably determined in a training or a learning phase using a training or a learning algorithm, including supervised or unsupervised. Upon completion of the training or the learning phase, initial predetermined values are determined and these may be updated via a learning algorithm. Accordingly, in embodiments, the constant matrices detailed above include values that are determined by a fuzzy neural network.

In another embodiment illustrated in FIG. 2b, the electromagnetic field inducing coils 81a, 82a, 83a, 84a, 85a, 86a can function as the impeller position sensors. In other words, the position detection system includes the electromagnetic field inducing coils 81a, 82a, 83a, 84a, 85a, 86a embedded in the housing 20.

In an exemplary embodiment, when one of more of the electromagnetic field inducing coils 81a, 82a, 83a, 84a, 85a, 86a are not being supplied with power to control movement of the impeller 51 then they may be employed to generate signals indicative of the relative position of the impeller 51 within the internal chamber 30 of the housing 20.

During commutation, two of the three upper electromagnetic field inducing coils 81a, 82a, 83a are active while the third is a sensing coil for Back emf. This allows the electronics to detect when the upper permanent magnets 62, 64, 66, 68 in the blades 60 of the impeller 51 cross one of the three coils 81a, 82a, 83a (zero crossing detection) and hence apply energy to one of the other two of the three coils 81a, 82a, 83a to pull and the other to push against any one or more of the permanent magnets 62, 64, 66, 68 in the blades 60.

Similarly, during commutation two of the three lower electromagnetic field inducing coils 84a, 85a, 86a are active while the third is a sensing coil for Back emf. This allows the electronics to detect when the lower permanent magnets 111, 112, 113, 114 in the blades 60 of the impeller 51 cross one of the three coils 84a, 85a, 86a (zero crossing detection) and hence apply energy to one the other two of the three coils 84a, 85a, 86a to pull and the other to push against any one or more of the lower permanent magnets 111, 112, 113, 114 in the blades 60.

Accordingly, embodiments of the invention are advantageous in that the electromagnetic field inducing coils 81a, 82a, 83a, 84a, 85a, 86a are operable as commutating coils for driving rotation of the impeller 51, for magnetically levitating the impeller 51 and controlling the position of the impeller in three axes X, Y, Z by controlling the current provided to each of the coils 81a, 82a, 83a, 84a, 85a, 86a and for operating as sensors for detecting in real time the relative position of the impeller 51 within the internal chamber 30 of the housing 20.

For example the measurement of the position of the impeller 51 is done by detecting the Back emf in one of the upper coils and in one of the lower coils. The difference between Back emf generated between the top coil and bottom coil at B is proportional to the imbalance of the impeller for that blade of the impeller at that time. This difference can also be detected by magnetic sensors placed around the top and bottom of the pump housing if there are problems with this sensitivity a dedicated magnetic sensor can be used such as a linear hall effect sensor.

Such embodiments of the invention are advantageous in that the electromagnetic field inducing coils 81a, 82a, 83a, 84a, 85a, 86a are operable for controlling the position of the rotor 50 within the housing in three dimensions and for driving rotation of the rotor 50 (i.e. commutation) thereby enabling a smaller less complicated pump 10.

### Method

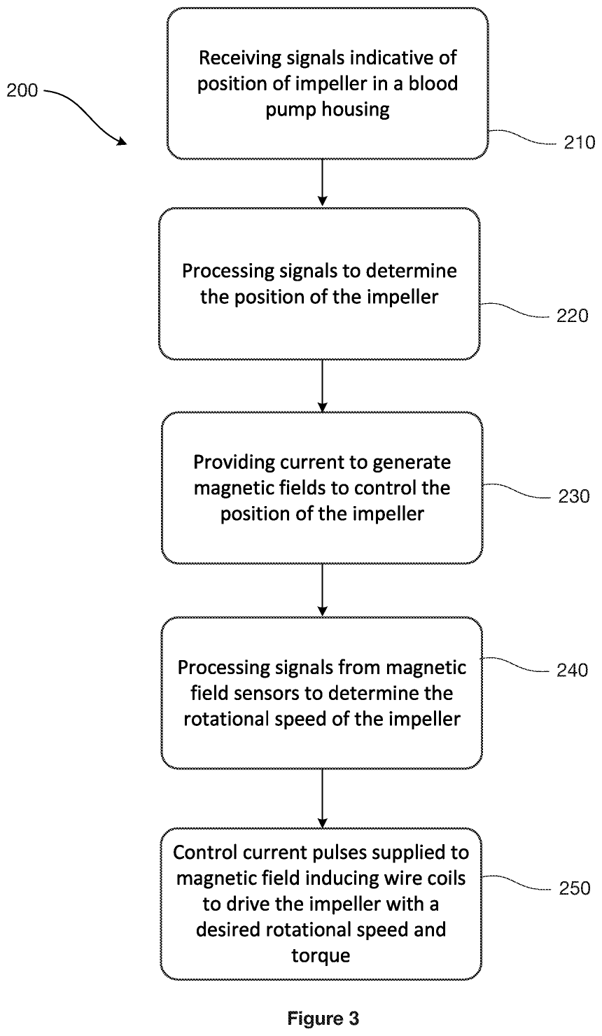

Embodiments of the present invention include methods for controlling a position of a rotor or impeller relative to an internal chamber of a housing of a blood pump. FIG. 3 illustrates steps in a method 200 in accordance with embodiments of the invention. For convenience, the embodiments of the method will be described with reference to the blood pump 10 illustrated in FIG. 1. However, it is to be appreciated that the method is not limited to the particular embodiment of the blood pump 10 described and illustrated therein.

The method 200 includes receiving in a controller signals from magnetic field sensors or from Back emf sensors indicative of the relative position of an impeller within a blood pump housing 210. The signals are processed in the controller to determine the relative position of the impeller within the blood pump housing 220.

An electrical current is then provided to one or more electromagnetic field inducing means embedded in the housing to thereby generate one or more magnetic fields 230. The one or more magnetic fields influence permanent magnets embedded in the rotor for controlling the position of the rotor relative to the internal chamber of the housing.

Embodiments of the method 200 further include processing the signals from magnetic field sensors or the Back emf sensors to determine the rotational speed of the impeller 240. The processor controls or adjusts current pulses supplied to electromagnetic field inducing wire coils to rotatably drive the impeller with a desired rotational speed and torque 250.

The invention may be susceptible to other modifications or mechanical equivalents without departing from the spirit or ambit of the invention disclosed herein.

1. A blood pump including:
a housing having an internal chamber, a blood inlet port and a blood outlet port;
a rotor including a plurality of blades and being adapted to rotate within the chamber wherein blood received into the chamber via the inlet port is directed by the blades of the rotor out of the chamber via the outlet port; and
a bearing system for controlling the position of the rotor relative to the internal chamber of the housing, the bearing system including:
one or more permanent magnets embedded in the rotor;
one or more electromagnetic field inducing means embedded in the housing;
wherein the permanent magnets embedded in the rotor are influenced by the electromagnetic field inducing means embedded in the housing for controlling the position of the rotor relative to the internal chamber of the housing and for driving rotation of the impeller within the chamber of the housing.

2. The blood pump of claim 1, wherein the permanent magnets and the electromagnetic field inducing means control the position of the rotor within the internal chamber of the housing in three dimensions.

3. The blood pump of claim 1, wherein the permanent magnets and the electromagnetic field inducing means control the position of the rotor within the internal chamber of the housing in an axial direction and in any direction in a plane normal to the axial direction.

4. The blood pump of claim 1, wherein the permanent magnets and the electromagnetic field inducing means are disposed radially about the rotor.

5. The blood pump of claim 1, wherein the permanent magnets are axially spaced apart from the electromagnetic field inducing means.

6. The blood pump of claim 1, wherein the permanent magnets and the electromagnetic field inducing means are oriented at an angle between the axis of rotation of the rotor and the normal to the axis of rotation of the rotor.

7. The blood pump of claim 6, wherein the permanent magnets and the electromagnetic field inducing means are oriented at an incline or at a decline to a horizontal plane that is perpendicular to a central longitudinal axis of the chamber.

8. The blood pump of claim 6, wherein the permanent magnets and the electromagnetic field inducing means are oriented at an incline or a decline angle of between about 20 to 50 degrees or between about 30 to 40 degrees or about 36 degrees.

9. The blood pump of claim 1, wherein the electromagnetic field inducing means includes one or more wire coils producing magnetic fields.

10. The blood pump of claim 12, wherein the electromagnetic field inducing means includes wire coils disposed radially about the central longitudinal axis of the housing at equal angularly spaced apart intervals.

11. The blood pump of claim 12, including upper electromagnetic field inducing means and lower electromagnetic field inducing means spaced apart in the direction of the central longitudinal axis of the housing.

12. The blood pump of claim 11, wherein the upper electromagnetic field inducing means and the lower electromagnetic field inducing means are respectively oriented at an incline and at a decline by equivalent angles from a horizontal plane of the impeller and are thereby oriented symmetrically about the horizontal plane.

13. The blood pump of claim 11, wherein the upper electromagnetic field inducing means are embedded in a downwardly sloping portion of an upper wall of the chamber and the lower electromagnetic field inducing means are embedded in an upwardly sloping portion of a lower wall of the chamber.

14. The blood pump of claim 9, wherein an electric current passed through each one of the wire coils is controlled independently to control the resulting magnetic fields produced thereby, wherein the interaction of the magnetic fields of the permanent magnets and of the wire coils induces forces acting between the impeller and the housing.

15. The blood pump of claim 14, wherein a resultant force between the impeller and the housing controls the relative position of the impeller within the chamber of the housing.

16. The blood pump of claim 14, wherein a resultant force between the impeller and the housing drives rotation of the impeller within the chamber of the housing.

17. The blood pump of claim 1, wherein the blood pump includes a rotor position detection system for detecting the position of the rotor relative to the internal chamber of the housing,

18. The blood pump of claim 17, wherein the position detection system includes:
one or more magnetic field sensors embedded in the housing;
wherein the magnetic field sensors are influenced by the permanent magnets embedded in the rotor for determining the position of the rotor relative to the internal chamber of the housing.

19. The blood pump of claim 18, wherein the magnetic field sensors are disposed radially about the rotor.

20. The blood pump of claim 18, wherein the magnetic field sensors are disposed at angularly spaced apart intervals.

21. The blood pump of claim 18, wherein six of the magnetic field sensors are disposed radially about the rotor at equal angularly spaced apart intervals.

22. The blood pump of claim 17, wherein the position detection system includes the electromagnetic field inducing means being operable for detecting the position of the impeller within the chamber of the housing.

23. The blood pump of claim 22, wherein the electromagnetic field inducing means are operable for detecting Back emf and thereby determining any imbalance of the impeller within the chamber of the housing.

24. The blood pump of claim 17, wherein the rotor position detection system detects the relative position of the rotor within the internal chamber of the housing in three dimensions including the axial direction and any direction in a plane normal to the axial direction.

25. The blood pump of claim 1, including a stator embedded in the housing comprised of the one or more electromagnetic field inducing means embedded in the housing, wherein the permanent magnets embedded in the rotor are influenced by a magnetic field generated by the one or more electromagnetic field inducing means embedded in the housing to thereby drive the rotation of the rotor within the chamber.

26. The blood pump of claim 1, wherein each one of the blades of the rotor includes at least one and preferably two of the embedded permanent magnets.

27. The blood pump of claim 26, wherein the embedded permanent magnets are oriented at an incline or at a decline to a horizontal plane that is perpendicular to a central longitudinal axis of the impeller, wherein the permanent magnets are oriented at an incline or a decline angle of between about 5 to 30 degrees or between about 10 to 20 degrees or about 15 degrees to the horizontal plane.

28. The blood pump of claim 27, wherein the one or more electromagnetic field inducing means are embedded in the housing in an orientation that is complementary to the orientation of the permanent magnets embedded in the rotor blades.

29. The blood pump of claim 1, wherein a controller is operable to control the speed of the pump to thereby control the output of the pump.

30. The blood pump of claim 29, wherein the controller is operable to control the magnetic fields induced by the one or more electromagnetic field inducing means embedded in the housing for controlling the position of the rotor relative to the internal chamber of the housing.

31. The blood pump of claim 29, wherein the controller receives a signal from the one or more magnetic field sensors or the electromagnetic field inducing means embedded in the housing and determines the position of the rotor relative to the internal chamber of the housing.

32. A method for controlling a position of a rotor relative to an internal chamber of a housing of a blood pump, the method including:
receiving in a controller signals from sensors indicative of the relative position of an impeller within a blood pump housing;
processing the signals in the controller to determine the relative position of the impeller within the blood pump housing; and
providing an electrical current to one or more electromagnetic field inducing means embedded in the housing to thereby generate one or more magnetic fields, wherein the one or more magnetic fields influence permanent magnets embedded in the rotor for controlling the position of the rotor relative to the internal chamber of the housing.

33. The method of claim 32, including selectively providing an electrical current to the one or more electromagnetic field inducing means embedded in the housing to selectively generate one or more magnetic fields for controlling the relative position of the rotor within the internal chamber of the housing in three dimensions.

34. The method of claim 33, wherein the three dimensions include a direction of the axis of rotation of the rotor and any direction in a plane normal to the axis of rotation of the rotor.

35. The method of claim 32, including receiving signals from a plurality of magnetic field sensors embedded in the housing that are induced by the permanent magnets embedded in the rotor and determining from the signals the position of the rotor relative to the internal chamber of the housing.
